(19) United States
(12) Patent Application Publication
(10) Pub. No.: US 2024/0424159 A1
(43) Pub. Date: Dec. 26, 2024

(54) CELLULOSIC FIBER BLENDS FOR DISINFECTING WIPES WITH IMPROVED TEAR STRENGTH

(71) Applicant: ECOLAB USA INC., Saint Paul, MN (US)

(72) Inventors: Emily Olson, Saint Paul, MN (US); Jonathan P. Fast, Saint Paul, MN (US); John Rogers, Saint Paul, MN (US)

(21) Appl. No.: 18/748,644

(22) Filed: Jun. 20, 2024

Related U.S. Application Data
(60) Provisional application No. 63/510,002, filed on Jun. 23, 2023.

(51) Int. Cl. A61L 2/18; A61L 101/36; D04H 3/013

(52) U.S. Cl. CPC A61L 2/18; D04H 3/013; A61L 2101/36; D10B 2201/22; D10B 2401/13

(57) ABSTRACT

Wipe compositions for cleaning, sanitizing and disinfecting with a sanitizing or disinfecting composition comprising a non-cationic surfactant chemistry and a substrate derived from cellulose or modified cellulose fibers comprising lyocell and viscose to provide an Elmendorf wet tear strength that approximates that of a 100% lyocell substrate of approximately the same basis weight (GSM) are disclosed. Methods of using the wipe compositions are also disclosed.

### CROSS REFERENCE TO RELATED APPLICATIONS

This application claims priority under 35 U.S.C. § 119 to provisional patent application U.S. Ser. No. 63/510,002, filed Jun. 23, 2023. The provisional patent application is herein incorporated by reference in its entirety, including without limitation: the specification, claims, and abstract, as well as any figures, tables, appendices, or drawings thereof.

### TECHNICAL FIELD

The disclosure relates generally to wipe compositions for sanitizing and disinfecting with a substrate derived from cellulose or modified cellulose fibers comprising lyocell and viscose to provide an Elmendorf wet tear strength that approximates that of a 100% lyocell substrate of approximately the same basis weight (GSM). Methods of using the wipe compositions are also provided.

### BACKGROUND

Cleaning wipes can be made from either synthetic or natural fibers. For example, cleaning wipes can be, for example, natural fibers such as cellulose or cellulose derivatives (e.g. viscose), or synthetic fibers such as polypropylene (PP) and polyester (PET). A majority of cleaning wipes commercially available utilize nonwoven substrates that are intended for single use and made from non-renewable, petrochemical sources. Such materials include thermoplastics (including polypropylene) which are not renewably sourced, biodegradable or compostable. These single use, disposable wipes create an environmental burden in light of the strong consumer demand for such products that are useful for various cleaning, disinfecting or sanitizing applications.

Although there has been consumer and industry interest in reducing environmental burdens, the development of reusable, compostable or materials from renewable sources has not met all commercial needs. For example, certain cleaning, disinfecting or sanitizing wipe products use cellulose substrates. However, these come with performance shortfalls. For example, cellulose substrates are not compatible with every chemical formulation. As a result, there remains a need for efficacious compositions saturated on wipe substrates made from renewable plant-based cellulose materials and to reduce wipes that are sources of single use plastics.

It is therefore an object of this disclosure to provide wipe compositions made from cellulose, derived from cellulose or other plant based substrates to limit the manufacture and use of single use plastics.

It is another object of this disclosure to formulate wipes that are biodegradable and/or compostable.

It is a further object of the disclosure to provide wipes that provide cleaning, sanitizing or disinfecting efficacy at least equivalent to existing wipes made from thermoplastic materials.

It is a still further object of the disclosure to provide wipes that provide the cleaning, sanitizing or disinfecting efficacy while also having improved tear strength.

Other objects, embodiments and advantages of this disclosure will be apparent to one skilled in the art in view of the following disclosure, the drawings, and the appended claims.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art with respect to tear strength of wipe compositions.

According to some aspects of the present disclosure, wipe compositions comprise: a substrate derived from cellulose or modified cellulose fibers comprising lyocell and viscose; and a sanitizing or disinfecting composition comprising a non-cationic surfactant chemistry and/or an organic acid; wherein the substrate is saturated with the disinfecting composition and has an Elmendorf wet tear strength that approximates that of a 100% lyocell substrate of approximately the same basis weight (GSM).

According to some additional aspects of the present disclosure, methods of using a wipe comprise: contacting a surface with a wipe composition as described herein, wherein the sanitizing or disinfecting composition provides at least a 3-log reduction against a target microbe within 10 minutes or less.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1 shows a graph measuring average tensile strength (dry) compared to percentage of lyocell in the cellulose derived wipe substrate as described in Example 1.

FIG. 2 shows a graph measuring average tensile strength (wet) compared to percentage of lyocell in the cellulose derived wipe substrate as described in Example 1, demonstrating a linear correlation between percentage lyocell and strength.

FIG. 3 shows a graph measuring average tear strength (wet) compared to percentage of lyocell in the cellulose derived wipe substrate as described in Example 2, demonstrating a non-linear correlation between percentage lyocell and Elmendorf wet tear strength.

FIG. 4 shows a graph measuring the tensile strength (wet) of substrates when saturated with an acid/anionic disinfecting chemistry as described in Example 4.

Various embodiments of the present disclosure will be described in detail with reference to the drawings, wherein like reference numerals represent like parts throughout the several views. Reference to various embodiments does not limit the scope of the disclosure. Figures represented herein are not limitations to the various embodiments according to the disclosure and are presented for exemplary illustration of the invention. An artisan of ordinary skill in the art need not view, within isolated figure(s), the near infinite number of distinct permutations of features described in the following detailed description to facilitate an understanding of the present invention.

### DETAILED DESCRIPTION

The present disclosure is not to be limited to that described herein, which can vary and are understood by skilled artisans. No features shown or described are essential to permit basic operation of the present disclosure unless otherwise indicated. It has been found that a substrate derived from cellulose or modified cellulose fibers comprising lyocell and viscose provides a stronger wet tear strength for compositions saturated with a sanitizing or disinfecting composition comprising a non-cationic surfactant chemistry.

It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

Unless defined otherwise, all technical and scientific terms used above have the same meaning as commonly understood by one of ordinary skill in the art to which embodiments of the present disclosure pertain.

The terms “invention” or “present invention” are not intended to refer to any single embodiment of the particular invention but encompass all possible embodiments as described in the specification and the claims.

The term “about,” as used herein, refers to variation in the numerical quantity that can occur, for example, through typical measuring techniques and equipment, with respect to any quantifiable variable, including, but not limited to, concentration, mass, volume, time, molecular weight, temperature, pH, molar ratios, log count of bacteria or viruses, and the like. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

As used herein, the term “antimicrobial” refers to a compound or composition that reduces and/or inactivates a microbial population, including, but not limited to bacteria, viruses, fungi, and algae within about 10 minutes or less, about 8 minutes or less, about 5 minutes or less, about 3 minutes or less, about 2 minutes or less, about 1 minute or less, or about 30 seconds or less. Preferably, the term antimicrobial refers to a composition that provides at least about a 3-log, 4-log, or 5-log reduction of a microbial population in about 10 minutes or less, about 8 minutes or less, about 5 minutes or less, about 3 minutes or less, about 2 minutes or less, or about 1 minute or less.

As used herein, the term “between” is inclusive of any endpoints noted relative to a described range.

As used herein, the term “cleaning” refers to a method used to facilitate or aid in soil removal, bleaching, microbial population reduction, and any combination thereof. As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism.

Terms characterizing sequential order, a position, and/or an orientation are not limiting and are only referenced according to the views presented.

As used herein, the term “disinfectant” refers to an agent that kills all vegetative cells including most recognized pathogenic microorganisms. In an embodiment, a disinfectant according to U.S. standards can use the procedure described in A.O.A.C. Use Dilution Methods, Official Methods of Analysis of the Association of Official Analytical Chemists, paragraph 955.14 and applicable sections, 15th Edition, 1990 (EPA Guideline 91-2). As used herein, the term “high level disinfection” or “high level disinfectant” refers to a compound or composition that kills substantially all organisms, except high levels of bacterial spores, and is affected with a chemical germicide cleared for marketing as a sterilant by the Food and Drug Administration. As used herein, the term “intermediate-level disinfection” or “intermediate level disinfectant” refers to a compound or composition that kills mycobacteria, most viruses, and bacteria with a chemical germicide registered as a tuberculocide by the Environmental Protection Agency (EPA). As used herein, the term “low-level disinfection” or “low level disinfectant” refers to a compound or composition that kills some viruses and bacteria with a chemical germicide registered as a hospital disinfectant by the EPA.

As used herein, the term “Elmendorf wet tear strength” refers to the measured grams of force (gf) required to tear a wet fiber wipe according to the methods described in the Example 2. In embodiments, the wipe compositions described herein have an Elmendorf wet tear strength that approximates that of a 100% lyocell substrate of approximately the same basis weight (GSM). A wet tear strength that that “approximates” that of a 100% lyocell substrate is within about 20%, or about 10% or about the same.

As used herein, the term “exemplary” refers to an example, an instance, or an illustration, and does not indicate a most preferred embodiment unless otherwise stated.

The phrase “free of” or similar phrases if used herein means that the composition comprises 0% of the stated component and refers to a composition where the component has not been intentionally added. However, it will be appreciated that such components may incidentally form thereafter, under some circumstances, or such component may be incidentally present, e.g., as an incidental contaminant.

The term “generally” encompasses both “about” and “substantially.”

The term “GSM” refers to the basis weight of a wipe composition measured in grams per square meter (gram/m²). Most commercially available wipe compositions have between about 20-100 GSM, or more often <40 GSM for synthetic substrates and >40 GSM for cellulose substrates, with variation between vendors expected.

The term “hard surface” refers to a solid, substantially non-flexible surface such as a counter top, tile, floor, wall, panel, window, plumbing fixture, kitchen and bathroom furniture, appliance, engine, circuit board, dish, mirror, window, monitor, touch screen, and thermostat. Hard surfaces are not limited by the material; for example, a hard surface can be glass, metal, tile, vinyl, linoleum, composite, wood, plastic, etc. Hard surfaces may include for example, health care surfaces and food processing surfaces.

As used herein, the phrase “health care surface” refers to a surface of an instrument, a device, a cart, a cage, furniture, a structure, a building, or the like that is employed as part of a health care activity. Examples of health care surfaces include surfaces of medical or dental instruments, of medical or dental devices, of electronic apparatus employed for monitoring patient health, and of floors, walls, or fixtures of structures in which health care occurs. Health care surfaces are found in hospital, surgical, infirmity, birthing, mortuary, and clinical diagnosis rooms. These surfaces can be those typified as “hard surfaces” (such as walls, floors, bed-pans, etc.), or fabric surfaces, e.g., knit, woven, and non-woven surfaces (such as surgical garments, draperies, bed linens, bandages, etc.), or patient-care equipment (such as respirators, diagnostic equipment, shunts, body scopes, wheel chairs, beds, etc.), or surgical and diagnostic equipment. Health care surfaces include articles and surfaces employed in animal health care.

As used herein, the term “instrument” refers to the various medical or dental instruments or devices that can benefit from cleaning with a composition as described herein. As used herein, the phrases “medical instrument,” “dental instrument,” “medical device,” “dental device,” “medical equipment,” or “dental equipment” refer to instruments, devices, tools, appliances, apparatus, and equipment used in medicine or dentistry. Such instruments, devices, and equipment can be cold sterilized, soaked or washed and then heat sterilized, or otherwise benefit from cleaning in a composition of the present disclosure. These various instruments, devices and equipment include, but are not limited to: diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressers, trocars, dilators, cages, glassware, tubing, catheters, cannulas, plugs, stents, scopes (e.g., endoscopes, stethoscopes, and arthroscopes) and related equipment, and the like, or combinations thereof.

As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism.

As used herein the term “polymer” refers to a molecular complex comprised of a more than ten monomeric units and generally includes, but is not limited to, homopolymers, copolymers, such as for example, block, graft, random and alternating copolymers, terpolymers, and higher “x”mers, further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

As used herein, the term “sanitizer” refers to an agent that reduces the number of bacterial contaminants to safe levels as judged by public health requirements. In an embodiment, sanitizers for use in this invention will provide at least a 99.9% reduction (3-log order reduction). These reductions can be evaluated using a procedure set out in Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2). According to this reference a sanitizer should provide a 99.9% reduction (3-log order reduction) within 30 seconds at room temperature, 25±2° C., against several test organisms. In some embodiments, a food contact sanitizer should provide a 99.999% reduction (5-log order reduction).

As used herein, the term “soil” or “stain” refers to any soil, including, but not limited to, non-polar oily and/or hydrophobic substances which may or may not contain particulate matter such as industrial soils, mineral clays, sand, natural mineral matter, carbon black, graphite, kaolin, environmental dust, and/or food based soils such as blood, proteinaceous soils, starchy soils, fatty soils, cellulosic soils, etc.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The term “substantially” refers to a great or significant extent. “Substantially” can thus refer to a plurality, majority, and/or a supermajority of said quantifiable variable, given proper context.

As used herein, the term “substantially free” refers to compositions completely lacking the component or having such a small amount of the component that the component does not affect the performance of the composition. The component may be present as an impurity or as a contaminant and shall be less than 0.5 wt-%. In another embodiment, the amount of the component is less than 0.1 wt-% and in yet another embodiment, the amount of component is less than 0.01 wt-%.

The term “surfactant” or “surface active agent” refers to an organic chemical that when added to a liquid changes the properties of that liquid at a surface.

The term “virus”, as used herein refers to a type of microorganism that can include both pathogenic and non-pathogenic viruses. Pathogenic viruses can be classified into two general types with respect to the viral structure: enveloped viruses and non-enveloped viruses. Some well-known enveloped viruses include herpes virus, influenza virus; paramyxovirus, respiratory syncytial virus, corona virus, HIV, hepatitis B virus, hepatitis C virus and SARS-CoV virus. Non-enveloped viruses, sometimes referred to as “naked” viruses, include the families Picornaviridae, Reoviridae, Caliciviridae, Adenoviridae and Parvoviridae. Members of these families include rhinovirus, poliovirus, adenovirus, hepatitis A virus, norovirus, papillomavirus, and rotavirus. It is known in the art that “enveloped” viruses are relatively sensitive and, thus, can be inactivated by commonly used disinfectants. In contrast, non-enveloped viruses are substantially more resistant to conventional disinfectants and are significantly more environmentally stable than enveloped viruses.

The term “weight percent,” wt-%, “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

### Wipe Substrates

The wipe compositions include a substrate derived from cellulosic fiber comprising lyocell and viscose and a sanitizing or disinfecting composition. In embodiments the cellulosic fiber is a plant-based fiber that is a natural fiber (also referred to as regenerative fiber). Examples of cellulosic fiber include pulp fibers, cellulose fibers and regenerated cellulose fibers (e.g. viscose or also referred to as rayon, and lyocell or also referred to as tencel). Pulp fibers are smaller, shorter fibers. Regenerated fibers are pulp that are prepared, dissolved, and extruded (i.e. reprocessed) to create longer fibers or a continuous fiber, where beneficially the chemical nature of the derivative is retained after the fiber formation process.

The wipe substrates referred to herein include cellulosic and modified cellulose fibers comprising lyocell and viscose, which is understood to include regenerative cellulose fibers comprising lyocell and viscose. Both viscose and lyocell are stronger than pulp. However, lyocell is known to be a stronger material than viscose due to its more uniform geometry and production method.

In preferred embodiments, the wipe substrate is derived from cellulosic fibers and comprises, consists of, or consists essentially of lyocell and viscose. Viscose is a treated pulp or cellulose fiber, such as treated with sodium hydroxide and carbon disulfide. Lyocell fibers are solvent extruded for increased mobility and alignment of the fiber chains, enhancing the crystalline character. Functionally this means that lyocell maintains its strength when wet, whereas viscose loses strength when wet. They are both renewable fibers that are commercially available in nonwoven forms from numerous suppliers. Both renewable fibers lyocell and viscose can have varying diameter, shape, elastic modulus, tensile strength and failure strain that is tunable during the fiber production.

In embodiments, the cellulosic fibers comprising lyocell and viscose have a fiber length between about 1-100 mm.

In preferred embodiments the wipe substrate is made up of at least 50%, at least 60%, at least 70%, at least 80%, at least 90%, at least 95%, or 100% of the cellulosic fibers comprising lyocell and viscose. In preferred embodiments the wipe substrate is made up of at least 90%, at least 95%, or 100% of the cellulosic fibers comprising lyocell and viscose. In embodiments, the wipe substrate is made up of about 100% of the cellulosic fibers lyocell and viscose.

In embodiments the wipe substrate and composition is substantially free or free of thermoplastic fibers and polymers, namely petrochemically derived thermoplastic fibers and polymers including for example, polypropylene (PP), polyvinyl chloride (PVC), polyethylene (PE), polyesters, including polyethylene terephthalate (PET), polyethers, polyacrylics, polyamides, polyesteramides, polyvinylalcohols, polystyrenes, and the like.

The terms “wipe” and “substrate” may be used interchangeably herein. In wipe substrate and composition is substantially free or free of synthetic fibers.

In embodiments the substrate wipe is a nonwoven substrate. Nonwoven substrates can be formed by any suitable technique as readily apparent to those of skill in the art, including for example where fibers are interlaid (often in a non-identifiable manner, i.e. nonwoven). Known techniques include for example, meltblown, spunbond, spunlaid, SMS (spunbond-meltblown-spunbond), co-formed, carded webs, thermal bonded, thermoformed, spunlace, hydroentangled, hydroembossed, needled, or chemically bonded. In embodiments where multiple layers of the fibers are used they may be bonded through any suitable technique as readily apparent to those of skill in the art, including for example hydroentangling.

Due to the cellulosic fiber derived materials the use of spunlace and hydroentangling may be preferred. Hydroentangling is a process where fibers are rearranged and entangled by fluid forces and creates frictional interlocking at the fiber level. Further consumer preference for soft feel of the wipe substrate can impact selection of the manufacturing techniques, such as disclosed in U.S. Publication No. 2022/0370303.

In an embodiment the wipe compositions comprise spunlace or hydroentangled cellulose or modified cellulose fibers comprising lyocell and viscose.

The wipe substrates described herein can also take various shapes and sizes, which are not intended to limit the scope of the disclosure herein. For example, wipes can include a single sheet of material, layers of material (e.g. 2 or more layers), material designed to conform to a particular shape, material suitable for affixing to a cleaning apparatus or implement (e.g. mop or other cleaning tool). Most commonly the substrate is in the form of a wipe.

In embodiments, the wipe is a single or multi use substrate. In some embodiments, beneficially the wipe compositions even when provided as single-use compositions are biodegradable or compostable.

The fibers have a basis weight (referring to the individual layers of the wipe substrate) measured in grams per square meter (GSM). In some embodiments the basis weight of the wipe substrate derived from cellulosic fibers and comprising lyocell and viscose is about 1 to about 200 GSM, or from about 20 to about 60 GSM. Exemplary nonwoven substrates are described in U.S. Patent Publication 2012/066852 and U.S. Patent Publication 2011/244199.

The wipe substrates and/or wipe compositions may be packaged in a variety of convenient containers or container systems. In embodiments, the container or container system includes a vessel or container for storage and dispensing, such as a flowpack container including a flexible film with wipe stack contained therein. In preferred embodiments the container or container system includes a lid to reduce spillage and/or evaporation of the sanitizing or disinfecting composition. In preferred embodiments the container or container system has a lid that allows the wipes to be pulled through an opening of the container that causes minimal leakage and no tearing of the wipe substrate. Preferably the wipes are packaged in rolls, stacks or piles made up of any number of wipes. Typically the container or container system houses from 10 to about 500 wipes. In embodiments, the wipe substrate may be perforated into individual use sizes that can be easily removed from the container or container system by a user.

### Cleaning Compositions

The wipe compositions include a sanitizing or disinfecting composition. In embodiments, the sanitizing or disinfecting composition is a non-reactive chemistry, including non-cationic active chemistry and/or organic acids.

The compositions include a non-cationic active chemistry, e.g. an anionic chemistry, that is suitable for use with the negatively charged substrate derived from cellulose or modified cellulose fibers. This is distinct from conventional antimicrobial wipe compositions which conventionally employ cationic quaternary ammonium compound as an antimicrobial agents which are positively charged and thereby bind to the negatively charged substrate derived from cellulose or modified cellulose fibers and can interfere with release of the chemistry (i.e. chemistry not fully released) for the cleaning, sanitizing and/or disinfecting properties on a surface. In embodiments, the compositions are substantially or free of cationic active chemistries, including for example, quaternary ammonium compounds, as well as peroxycarboxylic acid compositions, and/or oxidizing chemistries.

In embodiments, the sanitizing or disinfecting composition comprises an anionic active chemistry, namely a surfactant. In embodiments, the sanitizing or disinfecting composition comprises organic acid, such as citric acid. In embodiments, alpha hydroxycarboxylic acid, such as lactic acid, citric acid, tartaric acid, malic acid, gluconic acid, and the like; carboxylic acids, such as formic acid, acetic acid, propionic acid and the like; other common organic acids such as ascorbic acid, glutamic acid, levulinic acid, etc. could also be used. In some embodiments, the sanitizing or disinfecting composition the alpha hydroxycarboxylic acid does not include citric acid. In some embodiments, the sanitizing or disinfecting composition comprises a non-cationic active chemistry, namely a surfactant, and an organic acid. Any of the composition embodiments can further include solvents and optional additional functional ingredients.

In embodiments, the wipe compositions are preloaded with a sanitizing or disinfecting composition to provide a desired level of antimicrobial efficacy (e.g. sanitizing or disinfecting) based on a requisite log reduction in the population of a given target microbe. In other embodiments the wipe compositions can be saturated or loaded with the sanitizing or disinfecting composition at or shortly prior to an application of use thereof to provide a desired level of antimicrobial efficacy.

The sanitizing or disinfecting composition is loaded onto the substrate at a loading ratio of at least about 2:1 to about 8:1, or about 3:1 to about 5:1, by weight of the chemistry to the substrate.

The sanitizing or disinfecting composition comprises a non-cationic surfactant chemistry and a solvent. The composition can further comprise water. The non-cationic surfactant chemistry provides a negative charge that does not bind to the negatively charged substrate derived from cellulose or modified cellulose fibers comprising lyocell and viscose.

Although a focus of the wipe compositions are largely described in the context of sanitizing or disinfecting wipes, where the wipe compositions could alternatively be loaded with a cleaning component to provide general cleaning, without providing antimicrobial efficacy. Such wipe compositions can include water, surfactants, and/or additional functional ingredients.

In embodiments the composition has a pH less than about 5. Beneficially, the pH of the composition does not negatively interfere with the tensile and/or tear strength of the wipe composition. Without being limited to a mechanism of action the pH and composition components allow preloading of the composition on the wipe substrate weeks, months or year(s) before use without impacting the nature of the wipe composition.

### Non-Cationic Chemistries

The sanitizing or disinfecting composition comprises a non-cationic surfactant chemistry. In embodiments the non-cationic chemistry comprises an anionic surfactant. Anionic surfactants are surface active substances which are categorized by the negative charge on the hydrophile; or surfactants in which the hydrophilic section of the molecule carries no charge unless the pH is elevated to the pKa or above (e.g. carboxylic acids). Carboxylate, sulfonate, sulfate and phosphate are the polar (hydrophilic) solubilizing groups found in anionic surfactants. Of the cations (counter ions) associated with these polar groups, sodium, lithium and potassium impart water solubility; ammonium and substituted ammonium ions provide both water and oil solubility; and, calcium, barium, and magnesium promote oil solubility. Preferred anionic surfactants are an anionic sulfonated or sulfated surfactant.

In a preferred embodiment, the at least one surfactant is an anionic sulfonated surfactant. Anionic sulfonated surfactants suitable for use in the compositions also include alkyl sulfonates, the linear and branched primary and secondary alkyl sulfonates, and the aromatic sulfonates with or without substituents. In an aspect, sulfonates include sulfonated carboxylic acid esters. In an aspect, suitable alkyl sulfonate surfactants include linear or branched C8-C22 alkylbenzene sulfonates, C8-C22 alkyl sulfonates or C10-C22 alkyl sulfonates, or C8-C16 alkyl sulfonates. An exemplary alkylbenzene sulfonate is dodecylbenzene sulfonic acid (DDBSA). In an exemplary aspect, the anionic alkyl sulfonate surfactant is linear alkyl benzene sulfonic acid (LAS). In a preferred embodiment employing LAS as the anionic surfactant, the compositions are most effective at pH 3.5 or below. In a further embodiment, the anionic sulfonate surfactant may alternatively or additionally include diphenylated sulfonates, and/or sulfonated oleic acid.

Anionic sulfated surfactants suitable for use in the compositions also include alkyl ether sulfates, alkyl sulfates, the linear and branched primary and secondary alkyl sulfates, alkyl ethoxysulfates, fatty oleyl glycerol sulfates, alkyl phenol ethylene oxide ether sulfates, the C₅-C₁₇ acyl-N—(C₁-C₄ alkyl) and —N—(C₁-C₂ hydroxyalkyl) glucamine sulfates, and sulfates of alkylpolysaccharides such as the sulfates of alkylpolyglucoside, and the like. Also included are the alkyl sulfates, alkyl poly(ethyleneoxy) ether sulfates and aromatic poly(ethyleneoxy) sulfates such as the sulfates or condensation products of ethylene oxide and nonyl phenol (usually having 1 to 6 oxyethylene groups per molecule). In some cases, the alkylene oxide bridge can be propylene oxide rather than, or in addition to ethylene oxide.

Preferred anionic sulfonated or sulfated surfactants include, but are not limited to, C8-C22 alkylbenzene sulfonates, sulfonated oleic acid, sodium lauryl sulfate, sodium laureth sulfate, a sulfosuccinate, a secondary alkane sulfonate, or mixtures thereof.

In some embodiments the anionic surfactant is C6-24 alkyl sulphates; C6-24 alkyl aryl sulphates; C6-24 alkyl alkoxylated sulphates; C6-24 alkyl sulphonates, including paraffin sulphonates; C6-24 alkyl aryl sulphonates; C6-24 alkyl alkoxylated sulphonates; C6-C24 alkyl alkoxylated linear or branched diphenyl oxide disulphonates; naphthalene sulphonates; and mixtures thereof.

In another embodiment the anionic surfactant is a branched alkyl sulphate surfactant. Branched alkyl sulphates include an alkyl sulfate comprising a sulfate group and a carbon chain of preferably from 2 to 20, or from 2 to 16, or from 2 to 8 carbon atoms. The carbon chain of the branched alkyl sulfate comprises at least one branching group attached to the carbon chain. The branching group is selected from the group consisting of an alkyl group having from 1 to 20, from 1 to 10, or from 1 to 4 carbon atoms. The branching group may be located at any position along the alkyl chain of the branched alkyl sulfate.

Additional anionic surfactants can include anionic carboxylate surfactants, those which have a carboxylic acid or an alpha hydroxyl acid group. Anionic carboxylate surfactants suitable for use in the present compositions include carboxylic acids (and salts), such as alkanoic acids (and alkanoates), ester carboxylic acids (e.g. alkyl succinates), ether carboxylic acids, and the like. Such carboxylates include alkyl ethoxy carboxylates, alkyl aryl ethoxy carboxylates, alkyl polyethoxy polycarboxylate surfactants and soaps (e.g. alkyl carboxyls). Secondary carboxylates useful in the present compositions include those which contain a carboxyl unit connected to a secondary carbon. The secondary carbon can be in a ring structure, e.g. as in p-octyl benzoic acid, or as in alkyl-substituted cyclohexyl carboxylates. The secondary carboxylate surfactants typically contain no ether linkages, no ester linkages and no hydroxyl groups. Further, they typically lack nitrogen atoms in the head-group (amphiphilic portion). Suitable secondary soap surfactants typically contain 11-13 total carbon atoms, although more carbons atoms (e.g., up to 16) can be present. Suitable carboxylates also include acylamino acids (and salts), such as acylgluamates, acyl peptides, sarcosinates (e.g. N-acyl sarcosinates), taurates (e.g. N-acyl taurates and fatty acid amides of methyl tauride), and the like.

Suitable anionic surfactants include alkyl or alkylaryl ethoxy carboxylates of the following formula: R—O—(CH₂CH₂O)ₙ(CH₂)ₘ—CO₂X in which R is a C₈ to C₂₂ alkyl group or

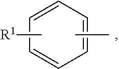

in which R¹ is a C₄-C₁₆ alkyl group; n is an integer of 1-20; m is an integer of 1-3; and X is a counter ion, such as hydrogen, sodium, potassium, lithium, ammonium, or an amine salt such as monoethanolamine, diethanolamine or triethanolamine. In some embodiments, n is an integer of 4 to 10 and m is 1. In some embodiments, R is a C₈-C₁₆ alkyl group. In some embodiments, R is a C₁₂-C₁₄ alkyl group, n is 4, and m is 1.

In other embodiments, R is

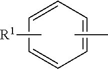

and R¹ is a C₆-C₁₂ alkyl group. In still yet other embodiments, R¹ is a C₉ alkyl group, n is 10 and m is 1.

Such alkyl and alkylaryl ethoxy carboxylates are commercially available. These ethoxy carboxylates are typically available as the acid forms, which can be readily converted to the anionic or salt form. Commercially available carboxylates include, Neodox 23-4, a C₁₂₋₁₃ alkyl polyethoxy carboxylic acid (Shell Chemical), and Emcol CNP-110, a C₉ alkylaryl polyethoxy carboxylic acid (Witco Chemical). Carboxylates are also available from Clariant, e.g. the product Sandopan® DTC, a C13 alkyl polyethoxy carboxylic acid.

Amphoteric, or ampholytic, surfactants contain both a basic and an acidic hydrophilic group and an organic hydrophobic group. These ionic entities may be any of anionic or cationic groups described herein for other types of surfactants. A basic nitrogen and an acidic carboxylate group are the typical functional groups employed as the basic and acidic hydrophilic groups. In a few surfactants, sulfonate, sulfate, phosphonate or phosphate provide the negative charge. Due to the pH of the system, it was found that many amphoteric surfactants, particularly those based on a carboxylic acid, were incompatible. In particular, it was found that the protonated portion of the carboxylic acid based amphoteric surfactants will complex with the anionic surfactant causing precipitation. Thus, limited amphoteric surfactants were found to be compatible with the system. Preferred amphoteric surfactants which can be included have a sulfate or sulfonate group.

Amphoteric surfactants can be broadly described as derivatives of aliphatic secondary and tertiary amines, in which the aliphatic radical may be straight chain or branched and wherein one of the aliphatic substituents contains from about 8 to 18 carbon atoms and one contains an anionic water solubilizing group, e.g., carboxy, sulfo, sulfato, phosphato, or phosphono. Amphoteric surfactants are subdivided into two major classes known to those of skill in the art and described in “Surfactant Encyclopedia” Cosmetics & Toiletries, Vol. 104 (2) 69-71 (1989), which is herein incorporated by reference in its entirety. The first class includes acyl/dialkyl ethylenediamine derivatives (e.g. 2-alkyl hydroxyethyl imidazoline derivatives) and their salts. The second class includes N-alkylamino acids and their salts. Some amphoteric surfactants can be envisioned as fitting into both classes.

Amphoteric surfactants can be synthesized by methods known to those of skill in the art. For example, 2-alkyl hydroxyethyl imidazoline is synthesized by condensation and ring closure of a long chain carboxylic acid (or a derivative) with dialkyl ethylenediamine. Commercial amphoteric surfactants are derivatized by subsequent hydrolysis and ring-opening of the imidazoline ring by alkylation—for example with chloroacetic acid or ethyl acetate. During alkylation, one or two carboxy-alkyl groups react to form a tertiary amine and an ether linkage with differing alkylating agents yielding different tertiary amines.

Long chain imidazole derivatives having application in the present invention generally have the general formula:

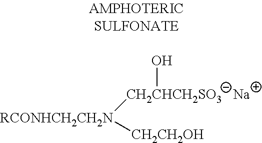

wherein R is an acyclic hydrophobic group containing from about 8 to 18 carbon atoms and M is a cation to neutralize the charge of the anion, generally sodium. Commercially prominent imidazoline-derived amphoterics that can be employed in the present compositions include for example: Cocoamphopropyl-sulfonate.

Zwitterionic surfactants can be thought of as a subset of the amphoteric surfactants and can include an anionic charge. Zwitterionic surfactants can be broadly described as derivatives of secondary and tertiary amines, derivatives of heterocyclic secondary and tertiary amines, or derivatives of quaternary ammonium, quaternary phosphonium or tertiary sulfonium compounds. Typically, a zwitterionic surfactant includes a positive charged quaternary ammonium or, in some cases, a sulfonium or phosphonium ion; and an alkyl group. Zwitterionics generally contain cationic and anionic groups which ionize to a nearly equal degree in the isoelectric region of the molecule and which can develop strong” inner-salt” attraction between positive-negative charge centers. Examples of such zwitterionic synthetic surfactants include derivatives of aliphatic quaternary ammonium, phosphonium, and sulfonium compounds, in which the aliphatic radicals can be straight chain or branched, and wherein one of the aliphatic substituents contains from 8 to 18 carbon atoms and one contains an anionic water solubilizing group, e.g., carboxy, sulfonate, sulfate, phosphate, or phosphonate.

Sultaine surfactants are exemplary zwitterionic surfactants for use herein. A general formula for these compounds is:

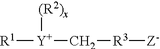

wherein R¹ contains an alkyl, alkenyl, or hydroxyalkyl radical of from 8 to 18 carbon atoms having from 0 to 10 ethylene oxide moieties and from 0 to 1 glyceryl moiety; Y is selected from the group consisting of nitrogen, phosphorus, and sulfur atoms; R² is an alkyl or monohydroxy alkyl group containing 1 to 3 carbon atoms; x is 1 when Y is a sulfur atom and 2 when Y is a nitrogen or phosphorus atom, R³ is an alkylene or hydroxy alkylene or hydroxy alkylene of from 1 to 4 carbon atoms and Z is a radical selected from the group consisting of sulfonate, sulfate, phosphonate, and phosphate groups.

Examples of zwitterionic surfactants having the structures listed above include: 5-[S-3-hydroxypropyl-S-hexadecylsulfonio]-3-hydroxypentane-1-sulfate; 3-[P,P-diethyl-P-3,6,9-trioxatetracosanephosphonio]-2-hydroxypropane-1-phosphate; 3-[N,N-dipropyl-N—3-dodecoxy-2-hydroxypropyl-ammonio]-propane-1-phosphonate; 3-(N,N-dimethyl-N-hexadecylammonio)-propane-1-sulfonate; 3-(N,N-dimethyl-N-hexadecylammonio)-2-hydroxy-propane-1-sulfonate; 3-[S-ethyl-S-(3-dodecoxy-2-hydroxypropyl) sulfonio]-propane-1-phosphate; 3-[P,P-dimethyl-P-dodecylphosphonio]-propane-1-phosphonate; and S [N,N-di(3-hydroxypropyl)-N-hexadecylammonio]-2-hydroxy-pentane-1-sulfate. The alkyl groups contained in said detergent surfactants can be straight or branched and saturated or unsaturated.

Sultaines useful in the present invention include those compounds having the formula (R(R¹)₂ N⁺R²SO³⁻, in which R is a C₆-C18 hydrocarbyl group, each R¹ is typically independently C₁-C₃ alkyl, e.g. methyl, and R² is a C₁-C₆ hydrocarbyl group, e.g. a C₁-C₃ alkylene or hydroxyalkylene group.

A typical listing of zwitterionic classes, and species of these surfactants, is given in U.S. Pat. No. 3,929,678 issued to Laughlin and Heuring on Dec. 30, 1975. Further examples are given in “Surface Active Agents and Detergents” (Vol. I and II by Schwartz, Perry and Berch). Each of these references is herein incorporated in their entirety.

In an embodiment, the compositions of the present invention include a betaine. For example, the compositions can include cocoamido propyl betaine.

The non-cationic surfactant chemistry comprises from about 0.01 wt-% to about 10 wt-%, from about 0.05 wt-% to about 10 wt-%, from about 0.1 wt-% to about 10 wt-%, from about 0.1 wt-% to about 9 wt-%, from about 0.1 wt-% to about 8 wt-%, from about 0.1 wt-% to about 7 wt-%, from about 0.1 wt-% to about 6 wt-%, from about 0.1 wt-% to about 5 wt-%, from about 0.1 wt-% to about 4 wt-%, from about 0.1 wt-% to about 3 wt-%, from about 0.1 wt-% to about 2 wt-%, or from about 0.1 wt-% to about 1 wt-%, or any range therebetween.

### Solvents

The sanitizing or disinfecting composition comprises a solvent. In some embodiments, the solvent is a hydrophobic oxygenated solvent. In some embodiments, solvents can include limited water-solubility alcohols.

In an aspect, a benzyl alcohol solvent and/or solvent system is employed. In a further aspect, a phenoxyethanol solvent and/or solvent system is employed. Without being limited to a particular mechanism of action, in some embodiments, the solvent provides a limited water solubility alcohol providing hydrophobicity that assists in comprising the virus. In an embodiment, the solvent has a solubility in water of preferably less than 15% water soluble, more preferably less than 8% water soluble, and most preferable less than 5% water soluble.

Additional suitable solvents and solvent systems may include one or more different solvents including aromatic alcohols, ether amines, amidines, 1,2-diols, esters, glycol ethers, and mixtures thereof. Representative glycol ether solvents may include aromatic glycol ether solvents, such as ethylene glycol phenyl ether (commercially available from Dow as Dowanol Eph) or diethylene glycol phenyl ether (commercially available as Dowanol DiEPh). Additional suitable glycol ether solvents may include, without limitation, Butyl CARBITOL™ acetate, Butyl CARBITOL™, Butyl CELLOSOLVE™ acetate, Butyl CELLOSOLVE™, Butyl DIPROPASOL™, Butyl PROPASOL™, CARBITOL™ PM-600, CARBITOL™ Low Gravity, CELLOSOLVE™ acetate, CELLOSOLVE™, DOWANOL PPH™, DOWANOL TPnB™, EEP™, FILMER IBT™, Hexyl CARBITOL™, Hexyl CELLOSOLVE™, Methyl CARBITOL™, Methyl CELLOSOLVE™ acetate, Methyl CELLOSOLVE™, Methyl DIPROPASOL™, Methyl PROPASOL acetate, Methyl PROPASOL™, Propyl CARBITOL™, Propyl CELLOSOLVE™, Propyl DIPROPASOL™, and/or Propyl PROPASOL™.

Additional suitable solvents may include 1,8-Diazabicyclo [5.4.0]undec-7-ene, or also may be referred to as 2,3,4,6,7,8,9,10-Octahydropyrimidol [1,2-a]azepine (or DBU), 2.5.7.10-tetraoxaundecante (TOU), acetamidophenol, acetanilide, acetophenone, 2-acetyl-1-methylpyrrole, ethyl hexyl glycerine, benzyl acetate, benzyl alcohol, methyl benzyl alcohol, alpha phenyl ethanol, benzyl benzoate, benzyloxyethanol, ethylene glycol phenyl ether, a propylene glycol, propylene glycol phenyl ether, amyl acetate, amyl alcohol, 3-butoxyethyl-2-propanol, butyl acetate, n-butyl propionate, cyclohexanone, diacetone alcohol, diethoxyethanol, diethylene glycol methyl ether, diisobutyl carbinol, diisobutyl ketone, dimethyl heptanol, dipropylene glycol tert-butyl ether, 2-ethylhexanol, ethyl propionate, ethylene glycol methyl ether acetate, hexanol, isobutanol, isobutyl acetate, isobutyl heptyl ketone, isophorone, isopropanol, isopropyl acetate, methanol, methyl amyl alcohol, methyl n-amyl ketone, 2-methyl-1-butanol, methyl ethyl ketone, methyl isobutyl ketone, 1-pentanol, n-pentyl propionate, 1-propanol, n-propyl acetate, n-propyl propionate, propylene glycol ethyl ether, tripropylene glycol methyl ether, dipropylene glycol n-propyl ether, tripropylene glycol n-propyl ether, dipropylene glycol n-butyl ether, tripropylene glycol n-butyl ether, diethylene glycol n-butyl ether acetate, diethylene glycol monobutyl ether, ethylene glycol n-butyl ether acetate, ethylene glycol monobutyl ether, dipropylene glycol monobutyl ether, propylene glycol monobutyl ether, ethyl 3-ethoxypropionate, 2,2,4-Trimethyl-1,3-Pentanediol Monoisobutyrate, diethylene glycol monohexyl ether, ethylene glycol monohexyl ether, diethylene glycol monomethyl ether, diethylene glycol monoethyl ether, ethylene glycol methyl ether acetate, ethylene glycol monomethyl ether, dipropylene glycol monomethyl ether, propylene glycol methyl ether acetate, propylene glycol monomethyl ether, diethylene glycol monopropyl ether, ethylene glycol monopropyl ether, dipropylene glycol monopropyl ether and propylene glycol monopropyl ether. Representative dialkyl carbonates include dimethyl carbonate, diethyl carbonate, dipropyl carbonate, diisopropyl carbonate and dibutyl carbonate. Representative oils include benzaldehyde, pinenes (alphas, betas, etc.), terpineols, terpinenes, carvone, cinnamealdehyde, borneol and its esters, citrals, ionenes, jasmine oil, limonene, dipentene, linalool and its esters. Representative dibasic esters include dimethyl adipate, dimethyl succinate, dimethyl glutarate, dimethyl malonate, diethyl adipate, diethyl succinate, diethyl glutarate, dibutyl succinate, dibutyl glutarate and products available under the trade designations DBE, DBE-3, DBE-4, DBE-5, DBE-6, DBE-9, DBE-IB, and DBE-ME from DuPont Nylon. Representative phthalate esters include dibutyl phthalate, diethylhexyl phthalate and diethyl phthalate. Additional solvents include glycerin alkyl ethers such as mono 2-ethyl hexyl glycerin ether, mono heptyl glycerin ether, di butyl glycerin ether, etc., as well as glycols such as 1,2 hexane diol and 1,2 octane diol.

The solvent(s), excluding water, comprises from about 0.1 wt-% to about 40 wt-%, from about 1 wt-% to about 40 wt-%, from about 1 wt-% to about 30 wt-%, from about 1 wt-% to about 25 wt-%, from about 1 wt-% to about 20 wt-%, from about 1 wt-% to about 15 wt-%, or from about 1 wt-% to about 10 wt-%, or any range therebetween.

### Water

The sanitizing or disinfecting composition comprises water in addition to another solvent. In such embodiments, water comprises from about 30 wt-% to about 99 wt-%, from about 40 wt-% to about 98 wt-%, or from about 50 wt-% to about 95 wt-%, or any range therebetween.

### Additional Functional Ingredients

The components of the sanitizing or disinfecting composition in the wipe composition can further be combined with various functional components suitable for uses disclosed herein. In some embodiments, the sanitizing or disinfecting compositions including the non-cationic surfactant chemistry and solvent make up a large amount, or even substantially all of the total weight of the compositions. For example, in some embodiments few or no additional functional ingredients are disposed therein.

In other embodiments, additional functional ingredients may be included in the sanitizing or disinfecting compositions. The functional ingredients provide desired properties and functionalities to the compositions. For the purpose of this application, the term “functional ingredient” includes a material that when dispersed or dissolved in a use and/or concentrate solution, such as an aqueous solution, provides a beneficial property in a particular use. Some particular examples of functional materials are discussed in more detail below, although the particular materials discussed are given by way of example only, and that a broad variety of other functional ingredients may be used. For example, many of the functional materials discussed below relate to materials used in cleaning. However, other embodiments may include functional ingredients for use in other applications.

In some embodiments, the sanitizing or disinfecting compositions may include solubility modifiers, dispersants, metal protecting agents, soil antiredeposition agents, stabilizing agents, corrosion inhibitors, builders/sequestrants/chelating agents, enzymes, aesthetic enhancing agents including emollients, fragrances and/or dyes, antimicrobial essential oils, additional disinfecting agents, additional rheology and/or solubility modifiers or thickeners, hydrotropes or couplers, pH modifiers (pH modifier may be alkaline or acidic and/or render a composition more alkaline or acidic), buffering agents (e.g. phosphonates, phosphonic acids, and/or phosphates), solvents, additional cleaning agents and the like.

In an embodiment the compositions further include a pH modifier. The compositions of the present application may include one or more pH adjusters/modifiers/buffers. The pH modifier may be alkaline or acidic and/or render a composition more alkaline or acidic. The pH modifier/buffer may be used to modify the pH and/or keep the pH of the composition in an optimal or preferred pKa range.

In an embodiment the compositions further include an alkaline agent, including for example hydroxide alkalinity.

Exemplary strong acids suitable for use modifying the pH of the compositions include methane sulfonic acid, sulfuric acid, sodium hydrogen sulfate, phosphoric acid, phosphonic acid, nitric acid, sulfamic acid, hydrochloric acid, trichloroacetic acid, trifluoroacetic acid, toluene sulfonic acid, glutamic acid, and the like; alkane sulfonic acid, such as methane sulfonic acid, ethane sulfonic acid, linear alkyl benzene sulfonic acid, xylene sulfonic acid, cumene sulfonic acid and the like. In a preferred aspect, the compositions include a strong acid having a pKa less than about 2.5 to beneficially provide the acidic use compositions having a pH less than about 4, or preferably less than about 3. In an embodiment, the compositions include a strong acid in combination with the non-cationic surfactant, and optionally include a weak acid.

Exemplary weak acids suitable for use modifying the pH of the compositions include alpha hydroxycarboxylic acid, such as lactic acid, citric acid, tartaric acid, malic acid, gluconic acid, and the like; carboxylic acids, such as formic acid, acetic acid, propionic acid and the like; other common organic acids such as ascorbic acid, glutamic acid, levulinic acid, etc. could also be used. In a preferred aspect, the compositions include a weak acid having a pKa greater than about 2.5 to beneficially provide the acidic use compositions having a pH less than about 4, or preferably less than about 3. In an embodiment, the compositions include a weak acid in combination with the non-cationic surfactant, and optionally include a strong acid.

In an embodiment the compositions further include a buffering agent. In a preferred embodiment, the composition employs a pH buffering agent with a pKa between about 2 and about 3. If a buffering agent is included in the compositions, it can be in any suitable amount to buffer the composition at a desired pH.

In an embodiment exemplary buffering agents include, but are not limited to, phosphonates, phosphonic acids, and/or phosphates. Exemplary buffering agents include a phosphonate salt(s) and/or a heterocyclic dicarboxylic acid, e.g., dipicolinic acid. In some embodiments, the buffering agent is a pyridine carboxylic acid-based buffers, such as picolinic acid and salts, pyridine-2,6-dicarboxylic acid and salts, and phosphonate based buffers, such as phosphoric acid and salts, pyrophosphoric acid and salts and most commonly 1-hydroxyethylidene-1,1-diphosphonic acid (HEDP) and salts. In other embodiments, the compositions and methods can comprise two or more buffering agents, e.g., HEDP and 2,6-pyridinedicarboxylic acid (DPA). Further, exemplary pH buffer agents include, but are not limited to, triethanol amine, imidazole, a carbonate salt, a phosphate salt, heterocyclic carboxylic acids, phosphonates, etc.

According to embodiments of the disclosure, the various additional functional ingredients may be provided in a composition in the amount from about 0 wt-% and about 40 wt-%, from about 0 wt-% and about 35 wt-%, from about 0 wt-% and about 30 wt-%, from about 0.01 wt-% and about 40 wt-%, from about 0.1 wt-% and about 40 wt-%, from about 1 wt-% and about 40 wt-%, from about 1 wt-% and about 30 wt-%, from about 1 wt-% and about 25 wt-%, or from about 1 wt-% and about 20 wt-%. In addition, without being limited according to the disclosure, all ranges recited are inclusive of the numbers defining the range and include each integer within the defined range.

### Methods of Use

The wipe compositions and methods of use thereof are particularly suited for consumer, industrial and institutional applications of use for cleaning, sanitizing or disinfecting. In some embodiments, the industrial and institutional applications can present more complex challenges in the types of surfaces in need of cleaning, sanitizing or disinfecting that require a wipe composition with improved tear strength. For example, various metal surfaces, sharp surfaces, and the like are often in need of cleaning, sanitizing or disinfecting in industrial and institutional applications where the wipe composition is subject to sharp surfaces that can conventionally cause a wipe to tear during use.

The wipe compositions are particularly useful for providing cleaning of a soil. The wipe compositions are also particularly useful for providing sanitizing or disinfecting against a target microbe within a given time period. The amount of time can be sufficient to allow, including from a few seconds to an hour, from about 30 seconds to about 15 minutes, or any range therebetween.

In embodiments, the sanitizing or disinfecting composition provides at least a 2-log, 3-log, 4-log, or 5-log reduction against a target microbe within about 10 minutes or less, about 8 minutes or less, about 5 minutes or less, about 3 minutes or less, about 2 minutes or less, or about 1 minute or less. As referred to herein, a 2-log reduction is equivalent to a 99% reduction, a 3-log reduction is equivalent to at least a 99.9% reduction, a 4-log reduction is equivalent to at least a 99.99% reduction, and a 5-log reduction is equivalent to at least a 99.999% reduction.

The methods may comprise a single step of applying the wipe composition onto the surface without direct physical removal, such as a rinse step. Instead, the wipe composition can be applied to the composition to both administer the sanitizing or disinfecting composition as well as remove any soils thereon a surface.

In some aspects, the methods can further include a precleaning step and thereafter followed by the applying of the compositions. The compositions and methods of use thereof can include treating cleaned or soiled surfaces with the wipe composition.

It is central to the compositions and methods described herein that the makeup of the wipe compositions do not negatively interfere antimicrobial efficacy of the products.

The wipe compositions are used in wiping a substrate or surface. Wiping includes any shearing action that the wipe composition undergoes while in contact with a target substrate or surface. Most commonly wiping is done by hand by a user to move the wipe composition across a surface. Wiping can further include any type of mechanical action as well.

The wipe compositions and methods of use thereof beneficially provide wet tear strength, as can be measured by Elmendorf wet tear strength that approximates (or is approximately equal to) that of a 100% lyocell substrate of approximately the same basis weight (GSM). As referred to herein a basis weight is approximately the same if +/−10% GSM. Providing pre-saturated wipes with strength in real use, i.e. under wet conditions, ensures tear resistance when wiping a variety of surfaces found in actual use, i.e. metal equipment, textured or sharp equipment, and the like.

### Embodiments

The present disclosure is further defined by the following numbered embodiments:

1. A wipe composition comprising: a substrate derived from cellulose or modified cellulose fibers comprising lyocell and viscose; and a sanitizing or disinfecting composition comprising a non-cationic surfactant chemistry and/or an organic acid; wherein the substrate is saturated with the disinfecting composition and has an Elmendorf wet tear strength that approximates that of a 100% lyocell substrate of approximately the same basis weight (GSM).

2. The composition of embodiment 1, wherein the substrate comprises at least about 10% lyocell, or at least about 30% lyocell.

3. The composition of embodiment 2, wherein the substrate comprises up to about 90% viscose, or up to about 70% viscose.

4. The composition of any one of embodiments 1-3, wherein the non-cationic chemistry comprises an anionic surfactant.

5. The composition of embodiment 4, wherein the anionic surfactant is an anionic sulfonated or sulfated surfactant.

6. The composition of any one of embodiments 1-5, wherein the organic acid comprises one or more of lactic acid, citric acid, tartaric acid, malic acid, and/or gluconic acid.

7. The composition of any one of embodiments 1-6, wherein the substrate is substantially free of thermoplastic and synthetic fibers.

8. The composition of any one of embodiments 1-7, wherein the substrate is substantially free of polyesters, polyolefins, and other synthetic fibers.

9. The composition of any one of embodiments 1-8, wherein the wipe is a non-woven composition.

10. The composition of any one of embodiments 1-9, wherein the sanitizing or disinfecting composition is loaded onto the substrate at a loading ratio of at least about 2:1 to about 8:1.

11. The composition of any one of embodiments 1-10, wherein the substrate has a basis weight between about 30-100 GSM, 30-80 GSM, or 40-80 GSM.

12. The composition of any one of embodiments 1-11, wherein the sanitizing or disinfecting composition does not include quaternary ammonium compounds, oxidizers (e.g. peroxycarboxylic acids), and/or reactive chemistries.

13. The composition of any one of embodiments 1-12, wherein the sanitizing or disinfecting composition further comprises an additional surfactant, a pH adjusting agent, a buffering agent, a dye, a fragrance, a wetting agent, a solubilizer, a coupling agent, a hydrotrope, or mixture thereof.

14. The composition of any one of embodiments 1-13, wherein the sanitizing or disinfecting composition has a pH less equal to or less than about 5 and does not negatively impact the tensile and/or tear strength of the wipe composition.

15. A method of using a wipe comprising: contacting a surface with a wipe composition according to any one of embodiments 1-14, wherein the sanitizing or disinfecting composition provides at least a 3-log reduction against a target microbe within 10 minutes or less.

16. The method of embodiment 15, wherein the substrate is pre-saturated with the disinfecting composition or wherein the substrate is saturated with the disinfecting composition at or before the time of use.

17. The method of any one of embodiments 15-16, wherein the contacting step is performed by wiping, mopping, or other otherwise contacting with the wipe.

18. The method of any one of embodiments 15-17, wherein the surface is a hard surface and the hard surface comprises a floor, a counter, a wall, a rail, a sink, a drain, a pipe, a fluid tank, a container, a ware, a bath, a shower, an instrument or combinations thereof.

19. The method of any one of embodiments 15-18, wherein the sanitizing or disinfecting composition provides at least a 3-log reduction against a target microbe within 5 minutes or less.

20. The method of any one of embodiments 15-19, wherein the wipe is a single or multi use substrate.

21. The method of any one of embodiments 15-20, wherein the wipe composition does not tear during use.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

The following wipe compositions in Table 1 were tested in the Examples and GSM was measured (n=10) to verify the vendor reported GSM as this feature is known to vary+/−10%. All measured GSM were within 10%. Testing wipes at approximately the same basis weight is important as increases in basis weight will increase reporting strengths (tensile) over lower basis weight substrates. All wipe compositions were obtained from a single vendor.

TABLE 1

| | Reported | Measured |
|---|---|---|
| Wipe Composition | GSM | GSM |
| 100% Lyocell | 60 | 55 |
| 50% Lyocell/50% Viscose | 62 | 58 |
| 50% Lyocell/50% Viscose | 58 | 60 |
| 30% Lyocell/70% Viscose | 62 | 63 |
| 100% Viscose | 55 | 58 |

The tensile strength of the wipes (dry and wet) was evaluated (n=5) as shown in Table 2 according to ASTM D5035-11 (reapproved 2019) for both dry and wet specimens. Dry tensile strength is the most common physical attribute on supplier data sheets for wipe substrates. Tensile strength measures the strength (Newtons (N)/50 mm) required to pull the wipe substrate apart from the ends while pulling linearly, which for the dry substrate, has no impact on the fiber blend.

TABLE 2

| | | | Avg MD | Avg MD |
|---|---|---|---|---|
| | Reported | Measured | tensile— | tensile— |
| Wipe Composition | GSM | GSM | dry | wet |
| 100% Lyocell | 60 | 55 | 114.3 | 117.9 |
| 50% Lyocell/50% Viscose | 62 | 58 | 120.7 | 93.4 |
| 50% Lyocell/50% Viscose | 58 | 60 | 91.2 | 75.2 |
| 30% Lyocell/70% Viscose | 62 | 63 | 118.2 | 81.9 |
| 100% Viscose | 55 | 58 | 96.1 | 51.5 |

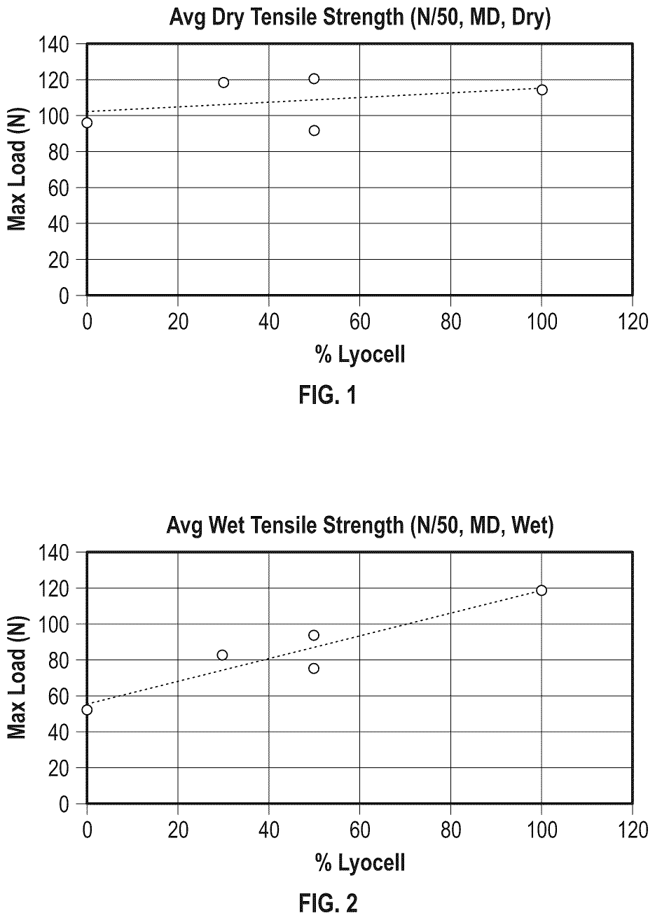

These results from Table 2 are further depicted in FIGS. 1-2, where FIG. 1 shows that there is no correlation between fiber blend and dry tensile strength, measured as Average Dry Tensile Strength (N/50, MD). Wet tensile strength is more challenging across wipe substrates. The data in FIG. 2 shows there is roughly a linear relationship between viscose and lyocell, i.e. blended substrates have tensile strength values that fall in the middle of the spectrum, measured as Average Wet Tensile Strength (N/50, MD).

This data confirms that according to tensile strength lyocell (as it is historically known to be) is a stronger wipe material than viscose. This is particularly true under wet conditions, as would be the case of wipes saturated with a chemistry composition such as the cleaning, sanitizing or disinfecting compositions described herein. The data shows that wet wipe substrates are more prone or susceptible to breaking under tensile (or elongating) conditions.

### Example 2

The tear strength (grams force) was tested according to Worldwide Strategic Partners Standard Test WSP 100.1.R³ (12) Standard Test Method for Tearing Strength of Nonwoven Fabrics by falling-Pendulum (Elmendorf) Apparatus at the various fiber blends shown in Table 3. The expectation was that lyocell would outperform viscose. As expected, lyocell was stronger to tearing than viscose, even though the listed GSM was lower. However, unexpectedly it was found that tear strength of lyocell/viscose blends did not increase linearly with lyocell content. Again, testing wipes at approximately the same basis weight is important as increases in basis weight will result in increased strength (tear strength measurements) over lower basis weight substrates.

TABLE 3

| | Measured | Wet Tear |
|---|---|---|
| Composition | GSM | (grams force) |
| 100% Lyocell | 55 | 552 |
| 50% Lyocell/50% Viscose | 58 | 498 |
| 50% Lyocell/50% Viscose | 60 | 470 |
| 30% Lyocell/70% Viscose | 63 | 470 |
| 100% Viscose | 58 | 238 |

Tear strength measures the average force required to propagate a single-rip tear starting from a cut in a nonwoven fabric using a falling-pendulum (Elmendorf) apparatus. This is more relevant to the use of the wipes, which can be subject to snagging on a surface or corner of a surface and then in need of increased tear strength, in particular while wet as would be the case in use. Consumers may accurately describe the tear strength as the ‘toughness’ or strength of the wipe.

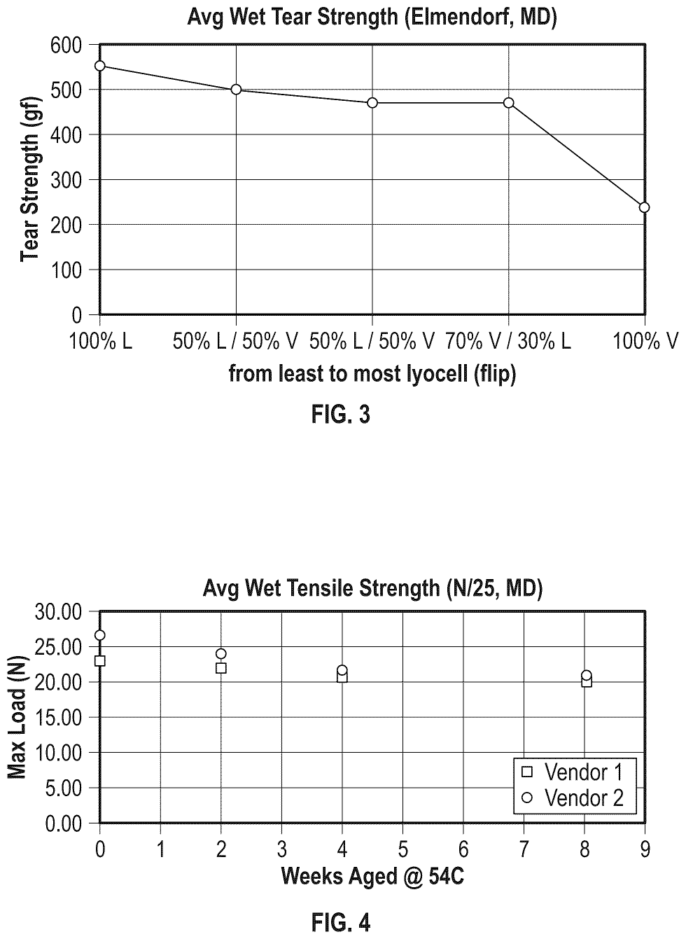

As shown in FIG. 3, the presence of lyocell in the blends (tested at ≥30% lyocell) provides similar strength to 100% lyocell. The measurement in FIG. 3 shows Average Wet Tear Strength (Elmendorf, MD). Although lyocell is historically known to provide a stronger wipe material than viscose, the tested blends of the wipes derived from cellulose show an improved tear strength or at least mimic the strength qualities of lyocell alone.

### Example 3

Additional data evaluating control (commercially-available) wipe products for wet tear strength was completed as described in Example 2 using the Elmendorf Wet Tear Strength methods. First a consumer wipe-both plastic and plant-based (cellulose) substrate-were compared as shown in Tables 4-5 (n=5 for all conditions). The examples tested in Table 5 are all synthetic wipe substrates.

TABLE 4

| | Substrate | Measured basis | MD tear—gf | |
|---|---|---|---|---|
| Control | type | weight (gsm) | Dry | Wet |
| Clorox plant-based | Cellulose | 44 | 254 | 153 |
| disinfecting wipes | | | | |
| Clorox disinfecting wipes | Synthetic | 47 | 420 | 462 |

TABLE 5

| | Measured basis | MD tear—gf |
|---|---|---|
| Control | weight (gsm) | Wet |
| Diversey Oxivir 1 | 37 | 372 |
| Clorox Healthcare Hydrogen Peroxide | 44 | 499 |
| Cleaner Disinfectant Wipes | | |
| PDI Super Sani-Cloth | 40 | 554 |
| | Average | 475 |

These results show that for the commercially-available control cellulose products have a significantly decreased wet tear strength, a challenge overcome by the wipe compositions and methods described herein using cellulose or modified cellulose fibers comprising lyocell and viscose. These lyocell blend substrates provide wet tear strength values that are approximately the same or similar to commercially-available synthetic wipes, in both consumer and healthcare environments. This provides a significant advantage over commercial products that are pre-saturated wipe compositions in need of wet tear strength as well as overcomes the need to replace synthetic wipe substrates as less desirable products in general, due to single use plastic products that are not environmentally-friendly. Beneficially, the lyocell blend substrates are a sustainable plant-based substrate that provides wet tear strength.

### Example 4

A trial was conducted to evaluate the tensile strength of 100% viscose substrates from two different vendors (Vendor 1 and Vendor 2). Tensile strength of the wipes was measured pursuant to ASTM D5035-11 (reapproved 2019) as described in Example 1 with modifications as described here for testing the substrates with the aging (i.e. testing over time).

The 100% viscose substrates were saturated with an acid/anionic surfactant (dodecyl benzene sulfonic acid, DDBSA) based disinfecting chemistry for evaluation of the acid-based composition on the shelf-stability in terms of tensile strength of the substrates. Prior to accelerated aging, 10 wipes were removed for time 0 tensile strength measurements.

Following the first measurement, the saturated rolls were then sealed and placed in a 54° C. oven to simulate accelerated aging. At each of the time points listed, the same roll was removed from the accelerated aging condition and 10 wipes were measured for wet tensile strength. Following this, the rolls were resealed and placed in the oven to repeat the process. The samples were each cut to 1′ width and then tested in the machine direction for tensile strength.

The tensile strength measurement utilized in this trial identified the maximum amount of force that the wipe can withstand prior to failing (pulling apart), referred to as max load in FIG. 4. This measurement of tensile strength is similar to tear strength, however the wipe is torn in a different way. Tensile strength pulls the wipe apart with the force in parallel, whereas tear uses a pendulum to tear the wipe in the perpendicular.

As reflected in FIG. 4, the data indicated that the Average Wet Tensile Strength (N/25, MD) measured in max load of Vendor 1 substrate decreased from 26 Newtons/meter² to 21 Newtons/meter² over the course of 8 weeks. By contrast, the Vendor 2 substrate demonstrated an initial tensile strength of 23 Newtons/meter² and decreased to 20 Newtons/meter² over the course of 8 weeks. The results confirmed that when the disinfecting composition utilized has a pH of less than 4, the pH did not negatively interfere with the tensile and/or tear strength of the wipe composition. The current example run with 100% viscose substrate is considered a control or baseline demonstration that pH did not negatively interfere with strength of the wipe substrate over the aged 8 week testing. The blended wipe substrates derived from cellulose or modified cellulose fibers comprising lyocell and viscose as described herein would provide at least the same 8 week aged wipe strength or an improvement thereof.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure. All publications discussed and/or referenced herein are incorporated herein in their entirety.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

What is claimed is:

1. A wipe composition comprising:
a substrate derived from cellulose or modified cellulose fibers comprising lyocell and viscose; and
a sanitizing or disinfecting composition comprising a non-cationic surfactant chemistry and/or an organic acid;
wherein the substrate is saturated with the disinfecting composition and has an Elmendorf wet tear strength that approximates that of a 100% lyocell substrate of approximately the same basis weight (GSM).

2. The composition of claim 1, wherein the substrate comprises at least about 10% lyocell, or at least about 30% lyocell.

3. The composition of claim 2, wherein the substrate comprises up to about 90% viscose.

4. The composition of claim 1, wherein the non-cationic chemistry comprises an anionic surfactant.

5. The composition of claim 4, wherein the anionic surfactant is an anionic sulfonated or sulfated surfactant.

6. The composition of claim 1, wherein the organic acid comprises one or more of lactic acid, citric acid, tartaric acid, malic acid, and/or gluconic acid.

7. The composition of claim 1, wherein the substrate is substantially free of thermoplastic and synthetic fibers.

8. The composition of claim 1, wherein the substrate is substantially free of polyesters, polyolefins, and other synthetic fibers.

9. The composition of claim 1, wherein the wipe is a non-woven composition.

10. The composition of claim 1, wherein the sanitizing or disinfecting composition is loaded onto the substrate at a loading ratio of at least about 2:1 to about 8:1.

11. The composition of claim 1, wherein the substrate has a basis weight between about 30-80 GSM.

12. The composition of claim 1, wherein the sanitizing or disinfecting composition does not include quaternary ammonium compounds, oxidizers, and/or reactive chemistries.

13. The composition of claim 1, wherein the sanitizing or disinfecting composition further comprises an additional surfactant, a pH adjusting agent, a buffering agent, a dye, a fragrance, a wetting agent, a solubilizer, a coupling agent, a hydrotrope, or mixture thereof.

14. The composition of claim 1, wherein the sanitizing or disinfecting composition has a pH less equal to or less than about 5 and does not negatively impact the tensile and/or tear strength of the wipe composition.

15. A method of using a wipe comprising:
contacting a surface with a wipe composition according to claim 1,
wherein the sanitizing or disinfecting composition provides at least a 3-log reduction against a target microbe within 10 minutes or less.

16. The method of claim 15, wherein the substrate is pre-saturated with the disinfecting composition or wherein the substrate is saturated with the disinfecting composition at or before the time of use.

17. The method of claim 15, wherein the contacting step is performed by wiping, mopping, or other otherwise contacting with the wipe.

18. The method of claim 15, wherein the surface is a hard surface and the hard surface comprises a floor, a counter, a wall, a rail, a sink, a drain, a pipe, a fluid tank, a container, a ware, a bath, a shower, an instrument or combinations thereof.

19. The method of claim 15, wherein the sanitizing or disinfecting composition provides at least a 3-log reduction against a target microbe within 5 minutes or less.

20. The method of claim 15, wherein the wipe is a single or multi use substrate, and wherein the wipe composition does not tear during use.
